(19) United States
(12) Patent Application Publication
(10) Pub. No.: US 2023/0355841 A1
(43) Pub. Date: Nov. 9, 2023

(54) BONE COMPOSITE AND COMPOSITIONS FOR PREPARING SAME

(71) Applicant: GRIFOLS WORLDWIDE OPERATIONS LIMITED, DUBLIN (IE)

(72) Inventors: Mª Isabel BRAVO CAMISON, PARETS DEL VALLES, BARCELONA (ES); Montserrat COSTA RIEROLA, PARETS DEL VALLES, BARCELONA (ES); Alba PEREZ PERARNAU, PARETS DEL VALLES, BARCELONA (ES)

(21) Appl. No.: 17/757,619

(22) PCT Filed: Dec. 18, 2020
(86) PCT No.: PCT/EP2020/087269

§ 371 Date: Jun. 17, 2022

(30) Foreign Application Priority Data
Dec. 18, 2019 (EP) 19383140.1

(51) Int. Cl. A61L 27/44; A61L 27/46; A61L 27/52; B33Y 70/10; B33Y 80/00; B33Y 10/00; B29C 64/106

(52) U.S. Cl. CPC A61L 27/446; A61L 27/46; A61L 27/52; B33Y 70/10; B33Y 80/00; B33Y 10/00; B29C 64/106; B29K 2995/0063; B29K 2105/0094

(57) ABSTRACT

Bone Composite and Compositions, particularly multicomponent or multipart compositions, are for the preparation of bone constructs for use in trauma, or cancer patients for example. The multipart compositions are based around combinations of fibrinogen, thrombin, hydrogels and calcium/phosphorous salts. The multipart compositions are capable of being printed to yield bone constructs using a 3D printing process to produce accurate and precise bone constructs of a desired geometry.

### FIELD OF THE INVENTION

The present invention relates to tissue fabrication. In particular, disclosed herein are compositions for 3D-printing of bone composites. The compositions allow bone composites to be printed with accuracy and fidelity from a design drawing. The present invention also relates to methods of 3D-printing the bone composites, and for bone composites obtainable from the compositions disclosed herein.

### BACKGROUND

Bone tissue grafts are widely used in orthopaedic, neuro-, maxillofacial, and dental surgery. While effective, the use of auto-, and allo-bone grafts has a number of limitations.

Autologous bone grafts are harvested from the patient, and as such they require additional surgery and present increased risks associated with its harvesting, such as risk of infection, blood loss and compromised structural integrity at the donor site.

Alternatively, load bearing allograft bones can be utilised as a substitute for autologous bone. Predominantly, they are extracted from cadavers and avoid the complexities and patient exposure associated with harvesting autologous bone from living donors. However, issues around sterility, suitability and long-term supply persist.

Prior to use, both allograft and autograft bone tissues need to be shaped for the specific application specified by the surgeon. Naturally, considerations about the sterility of the shaped bone grafts are paramount. In some circumstances, the implantation of viable bone grafts substitutes is limited by the available shapes and sizes extracted from living or deceased donors.

Synthetic bone substitute materials, and bone chips afford a more pliable raw material and can be readily remodelled and reshaped, however they do not immediately provide mechanical support to the patient. Such materials are often used to fill oddly shaped bone defects, however, they are not well suited for wrapping or resurfacing bone.

Use of human allograft bone tissue is very common in orthopaedic surgery. Invariably, the allograft tissue acts as a scaffold for compositions containing materials with osteoconductive, osteoinductive, and/or osteogenic properties. Suitable materials include proteins and/or stem cells.

International Patent Publication No. WO2012118843 addresses the above mentioned shortcomings with bone allografts and autografts by providing biocompatible modular scaffolds optionally coated with inter alia bone morphogenic protein.

Exploiting 3D printing technology and bioinks has also been suggested in the prior art as an alternative to ameliorate the downfalls of traditional allo-, and auto- bone grafts in bone replacement therapy. For example, International Patent Publication No. WO2018078130 discloses printable bioinks comprising cellulose nanofibrils, calcium-containing particles, and living cells such as mesenchymal stem cells, osteoblasts or induced pluripotent stem cells. The cellulose nanofibrils are critical to the structural integrity of the bone constructs.

Notwithstanding the state of the art there remains a need for further alternatives to the use of traditional allo-, and auto-bone grafts in bone replacement therapy. Such alternatives should be capable of being manufactured under controlled, sterile manufacturing conditions. Critical to this is the ability of the material to be formed in a precise and accurate manner to a surgeon's or other health professional's specification. Naturally, all such alternatives should exhibit mechanical integrity and be capable of providing structural and load bearing support once transplanted. Ideally, the material should be biocompatible and capable of being non-toxic to bioactive materials with osteoconductive, osteoinductive, and/or osteogenic properties.

### SUMMARY OF THE INVENTION

The words “comprises/comprising” and the words “having/including” when used herein with reference to the present invention are used to specify the presence of stated features, integers, steps or components but do not preclude the presence or addition of one or more other features, integers, steps, components or groups thereof.

It should be appreciated by those skilled in the art that the specific embodiments disclosed herein should not be read in isolation, and that the present specification intends for the disclosed embodiments to be read in combination with one another as opposed to individually. As such, each embodiment may serve as a basis for modifying or limiting other embodiments disclosed herein.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “10 to 100” should be interpreted to include not only the explicitly recited values of 10 to 100, but also include individual value and sub-ranges within the indicated range. Thus, included in this numerical range are individual values such as 10, 11, 12, 13 . . . 97, 98, 99, 100 and sub-ranges such as from 10 to 40, from 25 to 40 and 50 to 60, etc. This same principle applies to ranges reciting only one numerical value, such as “at least 10”. Furthermore, such an interpretation should apply regardless of the breadth of the range or the characteristics being described.

### Composition of the Invention

In a first aspect the present invention provides for a multi-part composition for 3-dimensional printing of a bone composite, the multi-part composition comprising:
i) a first part comprising fibrinogen in a pharmaceutically acceptable carrier;
ii) a second part comprising thrombin in a pharmaceutically acceptable carrier; and
iii) a third part comprising a pharmaceutically acceptable hydrogel mixed with at least one biocompatible inorganic material, the at least one inorganic material providing a source of at least one of calcium and phosphorous atoms,
wherein the third part has an apparent viscosity selected from the group consisting of: from about 1 to about 300 Pa·s at a shear rate of 0.1 51 as measured by rotational viscometer at 25° C. and 1 atm of pressure, and
   from about 1 to about 100 Pa·s at a shear rate of 1 s⁻¹ as measured by rotational viscometer at 25° C. and 1 atm of pressure,
and further wherein the third part is a standalone composition, or it is mixed with either the first part or the second part of the multipart composition, such that at least the first and second parts of the multi-part composition do not mix prior to printing by a 3-dimensional printing device.

As used herein, the term rotational viscometer refers to a device which works on the principle of measuring the force acting on a rotor (torque) when it rotates at a constant angular velocity (rotational speed) in a liquid. Rotational viscometers are used for measuring the viscosity of Newtonian (shear-independent viscosity) or non-Newtonian liquids (shear dependent viscosity or Apparent Viscosity). The third part of the multipart compositions of the invention are shear thinning, ie they have a shear dependent viscosity; as such it is necessary to specify the shear rate at which the viscosity is measured. For further information, the skilled person is directed to ASTM D2196-18e1: Standard Test Methods for Rheological Properties of Non-Newtonian Materials by Rotational Viscometer, the contents of which are incorporated herein by reference.

In one embodiment, the present invention provides for a two part composition in which:
the first part (fibrinogen) is mixed with the third part, or
the second part (thrombin) is mixed with the third part,
   and the remaining unmixed part is standalone.

In one embodiment, the two part composition of the invention comprises:
the first part (fibrinogen) mixed with the third part, and
the second part (thrombin) is standalone.

In a further embodiment, the present invention provides for a three part composition in which the first, second and third parts are standalone.

Advantageously, the compositions of the present invention allow accurate and precise printing of bone structures having defined, complex and often irregular shapes. As such, the compositions of the invention address an important unmet need around bone transplant shapes and sizes that can be problematic with cadaveric and autologous bone transplants.

The components of the present invention are all natural or biocompatible materials and as such present a low toxicity risk. Moreover, the individual parts of the composition of the present invention can be sterilised, thus reducing the risk of any associated infection once the bone construct is printed and transplanted into the body of a patient. Suitable, non-limiting, methods of sterilisation include sterile filtration, and radiation. Moreover, the compositions of the present invention can be printed in a sterile environment to yield sterile bone constructs.

Further advantageously, the compositions of the present invention afford bone constructs with desirable structural and mechanical properties capable of providing load bearing support. In one embodiment, the compositions of the present invention are absent any additional reinforcing materials. For example, the multi-part compositions of the present invention may be absent any polymeric or fibrous materials that add additional strength and mechanical support to the bone construct once printed. Non-limiting examples of polymeric materials that add strength and mechanical support may include poly(ethylene oxide)-poly(propylene oxide) copolymers. The fibrous materials may be of plant or animal origin. For example, the fibrous materials may be derived from cotton, flax, hemp, jute, bamboo, recycled wood, waste paper, cellulose. In particular, the compositions of the present invention may lack cellulose fibres.

Alternatively, in a further embodiment, the compositions of the present invention may contain additional reinforcing materials if necessary. For example, the multi-part compositions of the present invention may contain polymeric or fibrous materials that add additional strength and mechanical support to the bone construct once printed. Desirably, the polymeric and fibrous materials are biocompatible. For example, the fibrous materials may be of natural plant or animal origin. For example, the fibrous materials may be selected from the group consisting of cotton, flax, hemp, jute, bamboo, recycled wood, waste paper, and cellulose. Non-limiting examples of polymeric materials that add strength and mechanical support may include poly(ethylene oxide)-poly(propylene oxide) copolymers.

Further advantageously, the multipart compositions of the present invention are capable of providing viable, non-toxic environments to biologically active materials such as cells, proteins, and growth factors. Desirably, the biologically active materials such as cells and proteins possess osteoconductive, osteoinductive, and/or osteogenic properties. For example, the biologically active materials may be immature cells capable of differentiating into any cell type, bone morphogenic proteins, and combinations thereof. For example, the biologically active material may comprise human mesenchymal stem cells (hMSC), bone morphogenic proteins, and combinations thereof.

As used herein the terms osteoconductive, osteoinductive, and osteogenic are interrelated. In particular, osteogenesis is the process of bone formation. Osteoinduction is the process by which osteogenesis is induced and typically manifests in stimulating undifferentiated immature cells to become active osteoblasts. Osteoconductive is a term utilised to describe a graft material that serves as a scaffold for, and is conducive to new bone growth.

In one embodiment, the multi part composition of the present invention contains hMSC. The hMSC may be present within the third part of the multipart composition of the present invention. Suitably, the hMSC may be present at a concentration of between about 1*10³ to about 5*10¹⁰ hMSC per mL of pharmaceutically acceptable hydrogel. For example, the hMSC may be present at a concentration of between about 1*10⁴ to about 5*10⁹, such as about 1*10⁵ to about 5*10⁸, for example about 1*10⁵ to about 5*10⁷ hMSC per mL of pharmaceutically acceptable hydrogel. In one embodiment, the hMSC may be present at a concentration of between about 1*10⁶ to about 9*10⁶, hMSC per mL of pharmaceutically acceptable hydrogel.

In a further embodiment, the multipart compositions of the present invention may contain pharmacologically active agents, and may function as a depot or reservoir thereof. For example, the compositions of the present invention may contain chemotherapeutic agents, antineoplastic agents, anti-inflammatory agents, anti-infective agents and combinations thereof.

### Fibrinogen & Thrombin

Fibrinogen utilised within the present invention may be obtained and purified from human plasma. Alternatively, fibrinogen utilised within the present invention may be recombinant and obtained and purified from a recombinant process. In one embodiment, the first part of the multipart composition of the present invention may contain fibrinogen at a concentration between about 5 to about 200 mg/mL, for example from about 25 to about 175 mg/mL, such as about 50 to about 150 mg/mL, suitably from about 75 to about 125 mg/mL. In one embodiment, the first part of the multipart composition of the present invention may contain from about 75 to about 100 mg/mL of fibrinogen. The first part of the multipart composition of the present invention may contain about 80 mg/mL of fibrinogen.

Similarly, thrombin utilised within the present invention may be obtained and purified from human plasma. Alternatively, thrombin utilised within the present invention may be recombinant and obtained and purified from a recombinant process. In one embodiment, the second part of the multipart composition of the present invention may contain thrombin at a concentration between about 25 and 1500 IU/mL, for example from about 50 to about 1250 IU/mL, such as about 75 to about 1000 IU/mL, suitably from about 100 to about 750 IU/mL, for example from about 250 to about 750 IU/mL, such as from about 400 to about 600 IU/mL. In one embodiment, the second part of the multipart composition of the present invention may contain from about 450 to about 550 IU/mL of thrombin. The second part of the multipart composition of the present invention may contain about 500 IU/mL of thrombin.

The skilled person will appreciate that fibrinogen and thrombin are components in the human coagulation cascade. Thrombin acts on fibrinogen to yield the polymer fibrin, which results in a gelation type transition when the multipart compositions of the present invention are mixed. Conversion of fibrinogen (in the first part) to fibrin by the action of thrombin (in the second part) need not be 100% quantitative. The present invention also contemplates non-quantitative conversion in which a final bone construct may contain mixtures of fibrinogen, fibrin, and thrombin. Furthermore, in using the terms fibrinogen and thrombin the present specification includes within its scope derivatives of fibrinogen and thrombin that:
1. do not materially alter the interaction between the two proteins; or
2. do not materially alter the end fibrin product obtained.

Desirably, the first part of the multipart composition of the present invention comprises fibrinogen formulated in a pharmaceutically acceptable vehicle containing at least one amino acid. The amino acid may be selected from the group consisting of arginine, lysine, histidine, glutamic acid, aspartic acid, alanine, valine, leucine, isoleucine, and combinations thereof. For example, the amino acid may be selected from the group consisting of arginine, glutamic acid, isoleucine, and combinations thereof. In one embodiment, the first part of the multipart composition of the present invention comprises fibrinogen formulated in a pharmaceutically acceptable vehicle containing at least one amino acid, and a citrate buffer. In a further embodiment, the first part of the multipart composition may additional contain sodium salts, such as sodium chloride.

In yet a further embodiment, the first part of the multipart composition of the present invention comprises fibrinogen formulated in a pharmaceutically acceptable vehicle containing at least one amino acid, a citrate buffer, and a sodium salt such as sodium chloride. In this embodiment, the amino acid may be selected from the group consisting of arginine, lysine, histidine, glutamic acid, aspartic acid, alanine, valine, leucine, isoleucine, and combinations thereof. For example, the amino acid may be selected from the group consisting of arginine, glutamic acid, isoleucine, and combinations thereof.

Preferably, the second part of the multipart composition of the present invention comprises thrombin formulated in a pharmaceutically acceptable vehicle containing dissolved calcium. The pharmaceutically acceptable vehicle may contain soluble calcium salts. For example, the calcium salt may be calcium chloride. In a further embodiment, the second part of the multipart composition of the present invention may further contain albumin and/or at least one amino acid. For example, the second part of the multipart composition of the present invention may contain calcium salts, and albumin (in addition to thrombin). For example, the second part of the multipart composition of the present invention may contain calcium salts, and at least one amino acid (in addition to thrombin). In one embodiment, the second part of the multipart composition of the present invention may contain calcium salts, albumin and at least one amino acid with an uncharged side chain (in addition to thrombin). In one embodiment, the amino acid is selected from the group consisting of glycine, alanine, and combinations thereof. The calcium salt may be present at a concentration of about 0.01 to about 2.0 mM per IU of thrombin, for example about 0.01 to about 1.0 mM per IU of thrombin, such as about 0.01 to about 0.1 mM per IU of thrombin.

### Hydrogels

With reference to the third part of the multipart composition of the present invention the pharmaceutically acceptable hydrogel may be selected from the group consisting of a hydrophilic polysaccharide, a gelatin hydrogel, and combinations thereof. Within this specification references to the constituent hydrophilic polymer of the hydrogel, eg alginate, gelatin, etc. are to be construed as a reference to a hydrogel prepared using that hydrophilic polymer.

As used herein, the term hydrogel shall be construed as a material having a three-dimensional (3D) network of hydrophilic polymers that can swell in water and hold a large amount of water while maintaining the structure due to chemical or physical cross-linking of individual polymer chains. Hydrogels within the scope of the present invention may contain at least 10% w/w water, for example at least 20% w/w water, such as at least 30% w/w water. The Hydrogel may contain greater than 40% w/w water; in some embodiments the hydrogels may contain greater than 50% w/w water.

For example, the pharmaceutically acceptable hydrogel may be selected from the group consisting of an alginate, hyaluronic acid, gelatin and combinations thereof. In one embodiment, the pharmaceutically acceptable hydrogel may be selected from the group consisting of an alginate, hyaluronic acid and combinations thereof. In one embodiment, the pharmaceutically acceptable hydrogel is an alginate.

As used herein, the term alginate refers to a naturally occurring anionic polymer typically obtained from natural sources such as seaweed or bacteria. The material is biocompatible, has low toxicity, and can undergo mild gelation by addition of divalent cations such as Ca²⁺. Alginates are block copolymers containing blocks of (1,4)-linked β-D-mannuronate (M) and α-L-guluronate (G) residues. The blocks are composed of consecutive G residues (eg, GGGGGG), consecutive M residues (eg, MMMMMM), and alternating M and G residues (eg, GMGMGM). Alginates extracted from different sources differ in M and G contents as well as the length of each block. Without any intention of limiting the present invention by theory it is believed that the G-blocks of alginate are believed to participate in intermolecular cross-linking to form hydrogels.

Alginates within the scope of the present invention include simple chemical derivatives of the (1,4)-linked β-D-mannuronate (M) and α-L-guluronate (G) units, for example, without limitation ether, ester, carbonate and urethane derivatives.

Alginates within the scope of the present invention may have a molecular weight of between about 10,000 g/mol and about 500,000 g/mol. For example, alginates within the scope of the present invention may have a molecular weight of from about 40,000 g/mol to about 400,000 g/mol, such as about 50,000 g/mol to about 300,000 g/mol, suitably from about 60,000 g/mol to about 250,000 g/mol. In one embodiment, the alginate used in the present invention may have a molecular weight of about 75,000 to about 200,000 g/mol.

Alginates useful in the present invention may have a molecular weight of between about 10 kDa and about 500 kDa. For example, alginates within the scope of the present invention may have a molecular weight of from about 40 kDa to about 400 kDa, such as about 50 kDa to about 300 kDa, suitably from about 60 kDa to about 250 kDa. In one embodiment, the alginate used in the present invention may have a molecular weight of about 75 kDa to about 200 kDa.

Alginates useful within the composition of the present invention may have a G/M ratio of ≤2.5, such as ≤2, for example ≤1.5, such as ≤1.0, suitably ≤0.5. In one embodiment, alginates useful within the composition of the present invention may have a G/M ratio ≤1.

Alginates suitable for use within the composition of the present invention may have an apparent viscosity of about 1 to about 100 Pa·s, such as about 1 to about 80 Pa·s, for example about 1 to about 60 Pa·s, suitably about 3 to about 40 Pa·s, such as about 3 to about 30 Pa·s, for example about 4 to about 30 Pa·s, suitably about 4 to about 25 Pa·s at a shear rate of 10 s⁻¹ as measured by rotational viscometer at 25° C. and 1 atm of pressure.

Alginates suitable for use within the compositions of the present invention may have a molecular weight between 50,000 g/mol to about 300,000 g/mol, and a G/M ratio ≤2. Alginates suitable for use within the compositions of the present invention may have a molecular weight between 50,000 g/mol to about 300,000 g/mol, and an apparent viscosity of about 1 to about 60 Pa·s at a shear rate of 10 s⁻¹ as measured by rotational viscometer at 25° C. and 1 atm of pressure.

In another embodiment, alginates suitable for use within the compositions of the present invention may have a molecular weight between 75,000 g/mol to about 200,000 g/mol, and a G/M ratio ≤1. Alternatively, alginates suitable for use within the compositions of the present invention may have a molecular weight between 75,000 g/mol to about 200,000 g/mol, and an apparent viscosity of about 1 to about 60 Pa·s at a shear rate of 10 s⁻¹ as measured by rotational viscometer at 25° C. and 1 atm of pressure.

Alginates suitable for use within the compositions of the present invention may have a molecular weight between 50,000 g/mol to about 300,000 g/mol, a G/M ratio ≤2, and an apparent viscosity of about 1 to about 60 Pa·s at a shear rate of 10 s⁻¹ as measured by rotational viscometer at 25° C. and 1 atm of pressure.

In another embodiment, alginates suitable for use within the compositions of the present invention may have a molecular weight between 75,000 g/mol to about 200,000 g/mol, a G/M ratio ≤1, and an apparent viscosity of about 1 to about 60 Pa·s at a shear rate of 10 s⁻¹ as measured by rotational viscometer at 25° C. and 1 atm of pressure.

As used herein the term hyaluronic acid refers to a glycosaminoglycan polysaccharide comprising a repeating disaccharide of β4-glucuronic acid-β3-N-acetylglucosamine. Hyaluronic acid polymers are produced in commercial quantities by extracting the material from animal tissues or through recombinant expression in suitable organisms such as bacteria.

Hyaluronic acids used to prepare the hydrogels of the present invention may have a molecular weight of about 5 kDa to about 10,000 kDa, for example about 50 kDa to about 9000 kDa, such as about 100 kDa to about 8000 kDa, suitably about 500 kDa to about 7000 kDa, for example about 1000 kDa to about 5000 kDa, such as about 1000 kDa to about 4000 kDa, suitably about 1000 kDa to about 3000 kDa, for example about 1000 to about 2500 kDa, such as about 1500 kDa to about 2500 kDa.

Hyaluronic acids suitable for use within the composition of the present invention may have an apparent viscosity of about 1 to about 100 Pa·s, such as about 1 to about 80 Pa·s, for example about 1 to about 60 Pa·s, suitably about 3 to about 40 Pa·s, such as about 3 to about 30 Pa·s, for example about 4 to about 30 Pa·s, suitably about 4 to about 25 Pa·s at a shear rate of 10 s⁻¹ as measured by rotational viscometer at 25° C. and 1 atm of pressure.

Hyaluronic acids suitable for use in the compositions of the present invention may have a molecular weight of about 1000 kDa to about 4000 kDa, and an apparent viscosity of about 1 to about 60 Pa·s at a shear rate of 10 s⁻¹ as measured by rotational viscometer at 25° C. and 1 atm of pressure. For example, hyaluronic acids suitable for use in the compositions of the present invention may have a molecular weight of about 1000 kDa to about 2500 kDa, and an apparent viscosity of about 1 to about 60 Pa·s as measured by rotational viscometer at 25° C. and 1 atm of pressure.

As used herein, the term Gelatin refers to a mixture of peptides and proteins produced by thermal hydrolysis of collagen extracted from the skins, bones, tendon and white connectivity tissues of animals such as domesticated cattle, chicken, pigs, fish and even some insects. The source of gelatin from animals is hide and bone, and from vegetables is starch, alginate, pectin, agar and carrageenan. Gelatin is a heterogeneous mixture of high molecular weight polypeptides, which can swell and adsorb 5-10 times their weight of water to form a hydrogel. Gelatin is biodegradable, and non-toxic.

Gelatins suitable for use within the composition of the present invention may have an apparent viscosity of about 1 to about 100 Pa·s, such as about 1 to about 80 Pa·s, for example about 1 to about 60 Pa·s, suitably about 3 to about 40 Pa·s, such as about 3 to about 30 Pa·s, for example about 4 to about 30 Pa·s, suitably about 4 to about 25 Pa·s at a shear rate of 10 s⁻¹ as measured by rotational viscometer at 25° C. and 1 atm of pressure.

### Biocompatible Inorganic Material

With reference to the third part of the multipart composition of the present invention, the biocompatible inorganic material may provide a source of both calcium and phosphorous atoms. For example, the biocompatible inorganic material may be selected from the group consisting of bioglass, tricalcium phosphate, single-phase hydroxyapatite, biphasic hydroxyapatite-tricalcium phosphate, natural bone powder, and combinations thereof.

As used herein, the term bioglass refers to a calcium sodium phosphosilicate species.

In yet a further embodiment, the biocompatible inorganic material may be selected from the group consisting of tricalcium phosphate, single-phase hydroxyapatite, biphasic hydroxyapatite-tricalcium phosphate, and combinations thereof. The tricalcium phosphate utilised in the compositions of the present invention may be beta-tricalcium phosphate.

In order to be compatible with 3D printing processes and devices, preferably the biocompatible inorganic material has an average particle size of less than about 200 μm. For example the biocompatible inorganic material may have an average particle size of less than about 150 μm. Suitably, the biocompatible inorganic material may have an average particle size of less than about 100 μm. For example, the biocompatible inorganic material may have an average particle size of between about 50 and about 200 μm.

The biocompatible inorganic material may be incorporated into the third part of the multipart composition of the present invention at a concentration of between about 1 g to about 10 g of biocompatible inorganic material per 1 mL of hydrogel. For example, about 2 g to about 8 g, such as about 2 g to about 6 g of biocompatible inorganic material per 1 mL of hydrogel. In one embodiment, the biocompatible inorganic material may be incorporated into the third part of the multipart composition of the present invention at a concentration of between about 3 g to about 6 g of biocompatible inorganic material per 1 mL of hydrogel.

In some embodiments, the biocompatible inorganic materials of the present invention may be hydrated with an albumin solution prior to mixing with the pharmaceutically acceptable hydrogel. The albumin may be from any suitable source such as human, bovine or recombinant in origin. Preferably, the albumin is human albumin. The albumin solution may be saline based. In certain embodiments, the albumin solution may contain between about 0.01% to about 10% w/v of albumin. For example, between about 0.1% to about 5% w/v of albumin, such as between about 1% to about 3% w/v of albumin.

In one embodiment, the preferred biocompatible inorganic material utilised in the composition of the present invention is beta-tricalcium phosphate (Beta-TCP). The Beta-TCP may have a rhombohedral lattice with an R-3c space group. The Beta-TCP may be characterized by an X-ray powder diffraction pattern comprising unique peaks at °2θ (d value Å); angles of 17.0 (5.2), 21.9 (4.1), 25.8 (3.45), 27.8 (3.2), 29.65 (3.0), 31.0 (2.9), 32.45 (2.75), 34.4 (2.6), 46.9 (1.9), 48.0 (1.9), 48.4 (1.9), and 53.0 (1.7) when obtained with a Cu tube anode with K-alpha radiation.

The Beta-TCP may have a density of between about 2.9 g/cm³ and about 3.2 g/cm³ as determined by helium pycnometry. For example, the Beta-TCP may have a density of between about 2.9 g/cm³ and about 3.15 g/cm³ as determined by helium pycnometry. For example, the Beta-TCP may have a density of between about 2.9 g/cm³ and about 3.1 g/cm³ as determined by helium pycnometry In one embodiment, the Beta-TCP may have a density of between about 2.95 g/cm³ and about 3.15 g/cm³ as determined by helium pycnometry. In one embodiment, the Beta-TCP may have a density of between about 2.95 g/cm³ and about 3.1 g/cm³ as determined by helium pycnometry. In one embodiment, the Beta-TCP may have a density of between about 3.0 g/cm³ and about 3.1 g/cm³ as determined by helium pycnometry.

The Beta-TCP particles may have a d90 particle size distribution of not more than about 180 μm. In one embodiment, the Beta-TCP particles may have a d90 particle size distribution of not more than about 160 μm. In another embodiment, the Beta-TCP particles may have a d90 particle size distribution of not more than about 140 μm.

In one embodiment, the Beta-TCP used in the compositions of the present invention may be characterised by:
a d90 particle size distribution of not more than about 180 μm, and
a density of between about 2.9 g/cm³ and about 3.15 g/cm³ as determined by helium pycnometry.

In one embodiment, the Beta-TCP used in the compositions of the present invention may be characterised by:
a d90 particle size distribution of not more than about 160 μm, and
a density of between about 2.95 g/cm³ and about 3.15 g/cm³ as determined by helium pycnometry.

In one embodiment, the Beta-TCP used in the compositions of the present invention may be characterised by:
a d90 particle size distribution of not more than about 160 μm, and
a density of between about 2.95 g/cm³ and about 3.1 g/cm³ as determined by helium pycnometry.

In one embodiment, the Beta-TCP used in the compositions of the present invention may be characterised by:
an X-ray powder diffraction pattern comprising unique peaks at °2θ (d value Å); angles of 17.0 (5.2), 21.9 (4.1), 25.8 (3.45), 27.8 (3.2), 29.65 (3.0), 31.0 (2.9), 32.45 (2.75), 34.4 (2.6), 46.9 (1.9), 48.0 (1.9), 48.4 (1.9), and 53.0 (1.7) when obtained with a Cu tube anode with K-alpha radiation,
a density of between about 2.95 g/cm³ and about 3.15 g/cm³ as determined by helium pycnometry, and
a d90 particle size distribution of not more than about 180 μm.

In one embodiment, the Beta-TCP used in the compositions of the present invention may be characterised by:
an X-ray powder diffraction pattern comprising unique peaks at 2θ (d value Å); angles of 17.0 (5.2), 21.9 (4.1), 25.8 (3.45), 27.8 (3.2), 29.65 (3.0), 31.0 (2.9), 32.45 (2.75), 34.4 (2.6), 46.9 (1.9), 48.0 (1.9), 48.4 (1.9), and 53.0 (1.7) when obtained with a Cu tube anode with K-alpha radiation,
a density of between about 2.95 g/cm³ and about 3.1 g/cm³ as determined by helium pycnometry, and
a d90 particle size distribution of not more than about 160 μm.

### Third Part Composition

With reference to the third part of the composition of the invention, namely the mixture comprising the pharmaceutically acceptable hydrogel and the biocompatible inorganic material, in some embodiments the third part has an apparent viscosity selected from the group consisting of:
from about 30 to about 300 Pa·s at a shear rate of 0.1 s⁻¹ as measured by a rotational viscometer at 25° C. and 1 atm of pressure, and
from about 1 to about 75 Pa·s at a shear rate of 1 s⁻¹ as measured by a rotational viscometer at 25° C. and 1 atm of pressure.

In a further embodiment, the third part of the multipart composition of the invention may have an apparent viscosity selected from the group consisting of:
from about 30 to about 250 Pa·s at a shear rate of 0.1 s⁻¹ as measured by a rotational viscometer at 25° C. and 1 atm of pressure, and
from about 5 to about 75 Pa·s at a shear rate of 1 s⁻¹ as measured by a rotational viscometer at 25° C. and 1 atm of pressure.

For example, the third part of the multipart composition of the invention may have an apparent viscosity selected from the group consisting of:
from about 30 to about 200 Pa·s at a shear rate of 0.1 s⁻¹ as measured by a rotational viscometer at 25° C. and 1 atm of pressure, and
from about 5 to about 50 Pa·s at a shear rate of 1 s⁻¹ as measured by a rotational viscometer at 25° C. and 1 atm of pressure.

Suitably, the third part of the multipart composition of the invention may have an apparent viscosity selected from the group consisting of:
from about 50 to about 200 Pa·s at a shear rate of 0.1 s⁻¹ as measured by a rotational viscometer at 25° C. and 1 atm of pressure, and
from about 10 to about 45 Pa·s at a shear rate of 1 s⁻¹ as measured by a rotational viscometer at 25° C. and 1 atm of pressure.

In one embodiment, the third part of the multipart composition of the invention may have an apparent viscosity selected from the group consisting of:
from about 100 to about 300 Pa·s at a shear rate of 0.1 s⁻¹ as measured by a rotational viscometer at 25° C. and 1 atm of pressure, and
from about 1 to about 50 Pa·s at a shear rate of 1 s⁻¹ as measured by a rotational viscometer at 25° C. and 1 atm of pressure.

### Non-Limiting Embodiments

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier; and
iii) a third part comprising a pharmaceutically acceptable hydrogel mixed with at least one biocompatible inorganic material, the at least one inorganic material providing a source of at least one of calcium and phosphorous atoms,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   25 from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier; and
iii) a third part comprising an alginate hydrogel mixed with at least one biocompatible inorganic material, the at least one inorganic material providing a source of at least one of calcium and phosphorous atoms,
wherein the third part has an apparent viscosity selected from the group consisting of:
   from about 50 to about 250 Pa·s at a shear rate of 0.1 s⁻¹ as measured by a rotational viscometer at 25° C. and 1 atm of pressure, and
   from about 2 to about 50 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier; and
iii) a third part comprising a pharmaceutically acceptable hydrogel selected from the group consisting of an alginate, hyaluronic acid, gelatin, and combinations thereof mixed with at least one biocompatible inorganic material, the at least one inorganic material providing a source of at least one of calcium and phosphorous atoms,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier; and
iii) a third part comprising a pharmaceutically acceptable hydrogel selected from the group consisting of an alginate, hyaluronic acid, gelatin, and combinations thereof mixed with at least one biocompatible inorganic material selected from the group consisting of bioglass, tricalcium phosphate, single-phase hydroxyapatite, biphasic hydroxyapatite-tricalcium phosphate, natural bone powder, and combinations thereof,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel mixed with at least one biocompatible inorganic material selected from the group consisting of bioglass, tricalcium phosphate, single-phase hydroxyapatite, biphasic hydroxyapatite-tricalcium phosphate, natural bone powder, and combinations thereof,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel mixed with at least one biocompatible inorganic material, the at least one inorganic material providing a source of at least one of calcium and phosphorous atoms,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel selected from the group consisting of an alginate, hyaluronic acid, gelatin, and combinations thereof mixed with at least one biocompatible inorganic material, the at least one inorganic material providing a source of at least one of calcium and phosphorous atoms,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel selected from the group consisting of an alginate, hyaluronic acid, gelatin, and combinations thereof mixed with at least one biocompatible inorganic material selected from the group consisting of bioglass, tricalcium phosphate, single-phase hydroxyapatite, biphasic hydroxyapatite-tricalcium phosphate, natural bone powder, and combinations thereof,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel mixed with at least one biocompatible inorganic material, the at least one inorganic material providing a source of at least one of calcium and phosphorous atoms,

wherein the third part has an apparent viscosity selected from the group consisting of:

from about 50 to about 250 Pa·s at a shear rate of 0.1 s⁻¹ as measured by a rotational viscometer at 25° C. and 1 atm of pressure, and
from about 2 to about 50 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel mixed with at least one biocompatible inorganic material, the at least one inorganic material providing a source of at least one of calcium and phosphorous atoms,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel mixed with at least one biocompatible inorganic material, the at least one inorganic material providing a source of at least one of calcium and phosphorous atoms,
wherein the third part has an apparent viscosity selected from the group consisting of:
   from about 50 to about 250 Pa·s at a shear rate of 0.1 s⁻¹ as measured by a rotational viscometer at 25° C. and 1 atm of pressure, and
   from about 2 to about 50 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel selected from the group consisting of an alginate, hyaluronic acid, gelatin, and combinations thereof mixed with at least one biocompatible inorganic material, the at least one inorganic material providing a source of at least one of calcium and phosphorous atoms,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel selected from the group consisting of an alginate, hyaluronic acid, gelatin, and combinations thereof mixed with at least one biocompatible inorganic material selected from the group consisting of bioglass, tricalcium phosphate, single-phase hydroxyapatite, biphasic hydroxyapatite-tricalcium phosphate, natural bone powder, and combinations thereof,
wherein the third part has an apparent viscosity selected from the group consisting of:
   from about 1 to about 300 Pa·s at a shear rate of 0.1 s⁻¹ as measured by a rotational viscometer at 25° C. and 1 atm of pressure, and
   from about 1 to about 100 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel selected from the group consisting of an alginate, hyaluronic acid, gelatin, and combinations thereof mixed with at least one biocompatible inorganic material, the at least one inorganic material providing a source of at least one of calcium and phosphorous atoms,
wherein the third part has an apparent viscosity selected from the group consisting of:
   40 from about 50 to about 250 Pa·s at a shear rate of 0.1 s⁻¹ as measured by a rotational viscometer at 25° C. and 1 atm of pressure, and
   from about 2 to about 50 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle containing at least one amino acid selected from the group consisting of arginine, lysine, histidine, glutamic acid, aspartic acid, alanine, valine, leucine, isoleucine, and combinations thereof;
ii) a second part comprising thrombin in a pharmaceutically acceptable carrier containing dissolved calcium, and further excipient selected from the group consisting of albumin, an amino acid, and combinations thereof; and
iii) a third part comprising a pharmaceutically acceptable hydrogel selected from the group consisting of an alginate, hyaluronic acid, gelatin, and combinations thereof mixed with at least one biocompatible inorganic material selected from the group consisting of bioglass, tricalcium phosphate, single-phase hydroxyapatite, biphasic hydroxyapatite-tricalcium phosphate, natural bone powder, and combinations thereof,
wherein the third part has an apparent viscosity selected from the group consisting of:
   from about 50 to about 250 Pa·s at a shear rate of 0.1 s⁻¹ as measured by a rotational viscometer at 25° C. and 1 atm of pressure, and
from about 2 to about 50 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle;
ii) a second part comprising thrombin in a pharmaceutically acceptable carrier; and
iii) a third part comprising a pharmaceutically acceptable hydrogel selected from the group consisting of an alginate, hyaluronic acid, gelatin, and combinations thereof mixed with at least one biocompatible inorganic material, the at least one inorganic material providing a source of at least one of calcium and phosphorous atoms,
wherein the third part has an apparent viscosity selected from the group consisting of:
   from about 50 to about 250 Pa·s at a shear rate of 0.1 s⁻¹ as measured by a rotational viscometer at 25° C. and 1 atm of pressure, and
   from about 2 to about 50 Pa·s at a shear rate of 1 s⁻¹ as measured by a rotational viscometer at 25° C. and 1 atm of pressure.

In certain embodiments the multipart composition of the present invention may comprise or consist essentially of:
i) a first part comprising fibrinogen in a pharmaceutically acceptable vehicle;
ii) a second part comprising thrombin in a pharmaceutically acceptable carrier; and
iii) a third part comprising a pharmaceutically acceptable hydrogel selected from the group consisting of an alginate, hyaluronic acid, gelatin, and combinations thereof mixed with at least one biocompatible inorganic material selected from the group consisting of bioglass, tricalcium phosphate, single-phase hydroxyapatite, biphasic hydroxyapatite-tricalcium phosphate, natural bone powder, and combinations thereof,
wherein the third part has an apparent viscosity selected from the group consisting of:
   from about 50 to about 250 Pa·s at a shear rate of 0.1 s⁻¹ as measured by a rotational viscometer at 25° C. and 1 atm of pressure, and
from about 2 to about 50 Pa·s at a shear rate of 1 s⁻¹ as measured by a rotational viscometer at 25° C. and 1 atm of pressure.

In all the preceding embodiments, alginates suitable for use within the composition of the present invention may have an apparent viscosity of about 1 to about 100 Pa·s, such as about 1 to about 80 Pa·s, for example about 1 to about 60 Pa·s, suitably about 3 to about 40 Pa·s, such as about 3 to about 30 Pa·s, for example about 4 to about 30 Pa·s, suitably about 4 to about 25 Pa·s at a shear rate of 10 s⁻¹ as measured by rotational viscometer at 25° C. and 1 atm of pressure. Alginates suitable for use within the compositions of the present invention may have a molecular weight between 50,000 g/mol to about 300,000 g/mol, and a G/M ratio≤2. Alginates suitable for use within the compositions of the present invention may have a molecular weight between 50,000 g/mol to about 300,000 g/mol, and an apparent viscosity of about 1 to about 60 Pa·s at a shear rate of 10 s⁻¹ as measured by rotational viscometer at 25° C. and 1 atm of pressure.

In all the preceding embodiments, alginates suitable for use within the compositions of the present invention may have a molecular weight between 75,000 g/mol to about 200,000 g/mol, and a G/M ratio≤1. Alternatively, alginates suitable for use within the compositions of the present invention may have a molecular weight between 75,000 g/mol to about 200,000 g/mol, and an apparent viscosity about 1 to about 60 Pa·s at a shear rate of 10 s⁻¹ as measured by rotational viscometer at 25° C. and 1 atm of pressure. Alginates suitable for use within the compositions of the present invention may have a molecular weight between 50,000 g/mol to about 300,000 g/mol, a G/M ratio≤2, and an apparent viscosity about 1 to about 60 Pa·s at a shear rate of 10 s⁻¹ as measured by rotational viscometer at 25° C. and 1 atm of pressure. In another embodiment, alginates suitable for use within the compositions of the present invention may have a molecular weight between 75,000 g/mol to about 200,000 g/mol, a G/M ratio≤1, and an apparent viscosity about 1 to about 60 Pa·s at a shear rate of 10 s⁻¹ as measured by rotational viscometer at 25° C. and 1 atm of pressure.

### Bone Composite of the Invention

In a further aspect, the present invention provides for a bone composite obtainable from the multi-part composition of the present invention.

In certain embodiments, said biocompatible inorganic material is at a concentration of between about 5% w/w to about 60% w/w of the bone composite of the present invention. For example, at a concentration of between about 15% w/w to about 60% w/w, such as at a concentration of between about 25% w/w to about 60% w/w, suitably at a concentration of between about 35% w/w to about 60% w/w. In other embodiments, the biocompatible inorganic material is at a concentration selected from the group consisting of about 5% w/w to about 50% w/w, about 5% w/w to about 40% w/w, about 5% w/w to about 30% w/w, and about 10% w/w to about 25% w/w.

### Methods of the Invention

In yet a further aspect, the present invention provides for a method of preparing a bone composite, the method comprising the steps of:
providing a 3-dimensional printing device with the multi-part composition of the present invention;
printing the bone composite according to a determined design; and
optionally incubating the synthetic bone composite.

Naturally, owing to the multipart nature of the compositions of the invention various different permutations of the order in which the different parts are printed are possible and within the scope of the method of the present invention. For example, the method of the present invention for preparing a bone composite may comprise the 3-dimensional printing device printing;
i) a first layer comprising either fibrinogen or thrombin;
ii) a second layer comprising either fibrinogen or thrombin;
wherein if the first layer comprises fibrinogen the second layer comprises thrombin and vice versa,
iii) sequentially repeating steps i) and ii) n times, wherein n≥1, to generate an alternating layered structure;
iv) printing the third part comprising the hydrogel and biocompatible inorganic material on top of the alternating layered structure of step iii); and
v) optionally repeating steps i)-iv) as necessary to provide the bone composite.

With reference to the method of the present invention, in one embodiment the fibrinogen is printed first, and the thrombin is printed second as a layer on top of the fibrinogen.

For example, with reference to the embodiment of the method of the present invention outlined previously,
the first layer may have a defined volume of X,
the second layer may have a defined volume of Y,
steps i) and ii) may be repeated n times, wherein n≥1, to generate an alternating layered structure of volume nX+nY; and
wherein the third part comprising the hydrogel and biocompatible inorganic material is printed on top of the alternating layered structure, the third part having a volume Z.

In certain embodiments, the volumes X and Y are the same such that the volume nX=nY.

For example, in certain embodiments the ratio of Z to nX to nY (Z:nX:nY) is about 0.60-1.0:1:1. Suitably, the ratio of Z to nX to nY (Z:nX:nY) is about 0.80-1.0:1:1. In some embodiments, the ratio of Z to nX to nY (Z:nX:nY) is about 0.90-0.99:1:1. For example, the ratio of Z to nX to nY (Z:nX:nY) may be about 0.95:1:1. In yet a further embodiment, the 3-dimensional printer may print equal volumes of the first, second, and third parts of the multipart composition, such that the ratio of Z to nX to nY (Z:nX:nY) is about 1:1:1.

In other embodiments the ratio of Z to nX to nY (Z:nX:nY) is about 1-5:1:1. Suitably, the ratio of Z to nX to nY (Z:nX:nY) is about 2-4:1:1. In some embodiments, the ratio of Z to nX to nY (Z:nX:nY) is about 3-4:1:1. For example, the ratio of Z to nX to nY (Z:nX:nY) may be about 4:1:1.

In some embodiments 2≤n≤10.

In other embodiments of the method of the present invention for the preparation of a bone composite, the 3-dimensional printing device may print;
i) a first layer comprising the third part of the multipart composition of the present invention (ie, the hydrogel and biocompatible inorganic material);
ii) a second layer comprising either fibrinogen or thrombin;
iii) a third layer comprising either fibrinogen or thrombin;
wherein if the second layer comprises fibrinogen the third layer comprises thrombin and vice versa, and
iv) sequentially repeating steps ii) and iii) n times, wherein n≥1, to generate an alternating layered structure of fibrinogen and thrombin; and
v) optionally repeating steps i)-iv) as necessary to provide the bone composite.

For example, with reference to the embodiment of the method of the present invention outlined previously,
the first layer may have a defined volume of A,
the second layer may have a defined volume of B,
the third layer may have a defined volume of C,
wherein steps ii) and iii) may be repeated n times, wherein n≥1, to generate a layered structure of volume A+nB+nC.

In certain embodiments, the volumes B and C are the same such that the volume nB=nC.

For example, in certain embodiments the ratio of A to nB to nC (A:nB:nC) is about 1-5:1:1. Suitably, the ratio of A to nB to nC (A:nB:nC) is about 2-4:1:1. In some embodiments, the ratio of A to nB to nC (A:nB:nC) is about 3-4:1:1. For example, the ratio of A to nB to nC (A:nB:nC) may be about 4:1:1.

For example, in certain embodiments the ratio of A to nB to nC (A:nB:nC) is about 0.60-1.0:1:1. Suitably, the ratio of A to nB to nC (A:nB:nC) is about 0.80-1.0:1:1. In some embodiments, the ratio of A to nB to nC (A:nB:nC) is about 0.90-0.99:1:1. For example, the ratio of A to nB to nC (A:nB:nC) may be about 0.95:1:1. In further embodiments, the 3-dimensional printer may print equal volumes of the first, second, and third parts of the multipart composition, such that the ratio of A to nB to nC (A:nB:nC) is about 1:1:1.

In some embodiments 2≤n≤10.

For two-part compositions of the present invention, the method of the present invention may comprise the 3-dimensional printing device:
i) printing the first part of the composition comprising either fibrinogen or thrombin;
ii) repeating step i) n times, wherein n≥1, to generate a layered structure comprising only one of fibrinogen or thrombin;
iii) printing the second part comprising the other of fibrinogen or thrombin mixed with the pharmaceutically acceptable hydrogel and the biocompatible inorganic material on top of the layered structure generated in step ii);
iv) optionally repeating steps i)-iii) as necessary to provide the bone composite.

Alternatively, for two-part compositions of the present invention, the method of the present invention may comprise the 3-dimensional printing device:
i) printing the second part comprising one of fibrinogen or thrombin mixed with the pharmaceutically acceptable hydrogel and the biocompatible inorganic material;
ii) printing the first part comprising the other of fibrinogen or thrombin on top of the part printed in step i);
iii) repeating step ii) n times, wherein n≥1, to generate a layered structure
iv) optionally repeating steps i)-iii) as necessary to provide the bone composite.

With respect to the embodiments concerning printing the two-part compositions of the present invention the ratio of the volume of the first part printed to the volume of the second part printed (1^{st} part:2^{nd} part) may be about 1:1-8, for example about 1:6, such as about 1:4, suitably about 1:2, for example about 1:1. In certain embodiments, the ratio of the volume of the first part printed to the volume of the second part printed is 1:2.

In a further aspect, the present invention provides for a bone composite obtainable from the method of the present invention.

### Other Aspects of the Invention

In yet a further aspect, the present invention provides for a method of treating a bone injury, or bone defect in a patient in need thereof comprising the step of implanting the bone composite of the present invention into the patient in need thereof.

Further aspects of the present invention relate to the use of the bone composite of the present invention in the treatment of a bone injury, or a bone defect. The bone composite of the invention may find particular use in the treatment of trauma, and/or cancer patients.

In yet another aspect, the present invention provides for a kit comprising
a 3-dimensional printer, and
the multi-part composition of the present invention.

### BRIEF DESCRIPTION OF THE DRAWINGS

Additional features and advantages of the present invention will be made clearer in the appended drawings, in which:

FIG. 1 illustrates a series of print tests for the three part compositions of the present invention. A structure of defined geometry was printed and the results of the different compositions are outlined in the grid array shown;

FIG. 2 illustrates a plot of hMSC cell proliferation in the compositions of the present invention as measured by a ATP Cell Proliferation Assay (Promega);

FIG. 3 depicts a graphic of hMSC cell viability in the compositions of the present invention as measured using the LIVE/DEAD Cell Viability Assay (Life Technologies);

FIG. 4 shows the results of a hMSC osteogenic cell differentiation assay for the compositions of the present invention; and

FIG. 5 illustrates a series of print tests for two part compositions of the present invention.

### DETAILED EXAMPLES OF THE INVENTION

It should be readily apparent to one of ordinary skill in the art that the examples disclosed herein below represent generalised examples only, and that other arrangements and methods capable of reproducing the invention are possible and are embraced by the present invention.

### EXAMPLE 1

### Printing Objects of a Defined Geometry Using a Three-Part Composition of the Present Invention

Advantageously, the compositions of the present invention allow accurate and precise printing of structures having defined and irregular shapes. Outlined below are a series of tests illustrating the performance of a number of multipart compositions of the present invention. The compositions were loaded into a 3D printing device and a computer design drawing provided the shape of interest. The operation of a 3D printing device is within the normal skill and ability of a person of ordinary skill in the art.

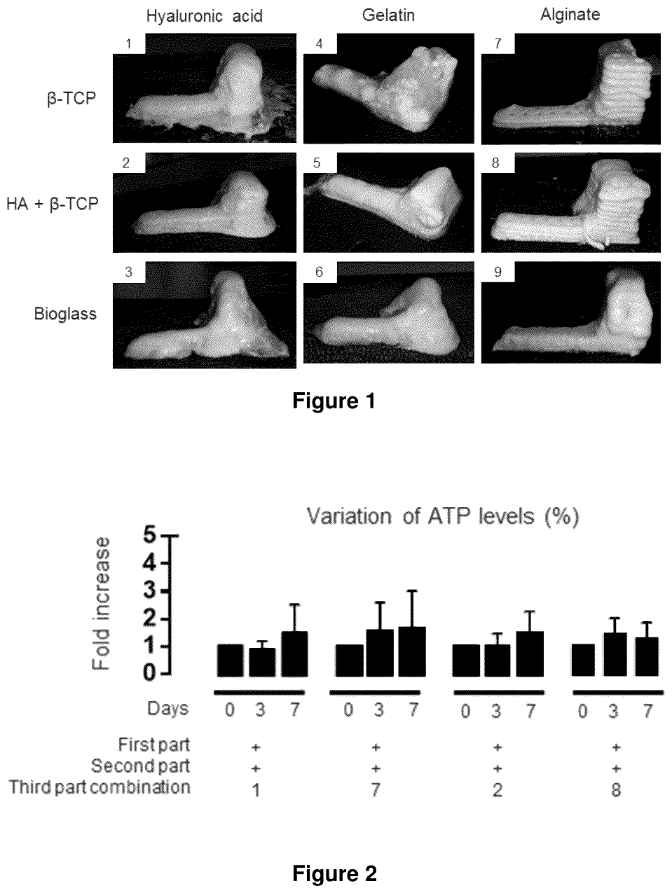

The components of the three-part compositions of the present invention are outlined below. All the bone constructs derived from the multipart compositions of the invention and illustrated in FIG. 1 contained the same fibrinogen component (first part) and thrombin component (second part), which are outlined qualitatively in Table 1 below.

TABLE 1

| Fibrinogen Component | Thrombin Component |
|---|---|
| Human Fibrinogen (80 mg/mL) | Human Thrombin (500 IU/mL) |
| Sodium citrate dihydrated | Calcium chloride |
| Sodium chloride | Human albumin |
| Arginine | Sodium chloride |
| Isoleucine | Glycine |
| Glutamic acid, monosodium | Water for injections |
| Water for injections | |

The surgical sealant product VERASEAL, manufactured and marketed by Grifols under Marketing Authorisation No. EU/1/17/1239/001-004 was the source of the fibrinogen and thrombin components utilised in all the experiments outlined in Examples 1-5. The product is also marketed as FIBRIN SEALANT in the United States of America by Grifols under Biologics License Number (BLN) 125640.

The hydrogel compositions were subsequently prepared using standard methodologies in the concentrations outlined in Table 2. For example, sodium alginate [(75-200 kDa, G/M ratio≤1, viscosity 20-200 mPa*s (PRONOVA UP LVM, DuPont)] 5 g was weighed out in a laminar flow cabinet. MilliQ water (100 mL) was heated to 50° C. and the sodium alginate was added with mixing in the laminar flow cabinet. The water was kept at a constant temperature of 50° C. until the sodium alginate powder was no longer visible (approx. 1 hour). The resulting solution was covered to maintain sterility and cooled to approximately 6° C. to accelerate the gelation process.

HYALUBRIX was utilised directly from the syringe.

TABLE 2

| Polymer | Concentration |
|---|---|
| Sodium alginate (75-200 kDa, G/M ratio ≤ 1, | 5 g/100 mL water |
| viscosity 140 mPa*s): PRONOVA ® UP LVM, | |
| DuPont | |
| Hyaluronic acid Sodium (1500-2000 kDa): | 1.5 g/100 mL water |
| HYALUBRIX, Fidia Pharma | |
| Gelatin (Viscosity 2.75-3.75 mPa*s, | 2 g/100 mL water |
| pH 4.5-5.5): Rousselot 250 PS, Rousselot | |

Table 3 outlines the constituents of, and their concentrations in the third part of the multipart composition of the present invention. Prior to their incorporation into the hydrogel, the biocompatible inorganic materials (Bioglass, TCP, etc.) in the weights indicated in Table 3, were hydrated with 8 mL of an albumin hydrating solution and manually mixed. The albumin hydrating solution consisted of human serum albumin at a concentration of 20 mg/mL in a saline vehicle. Both the saline and human serum albumin [ALBUTEIN] were obtained from Grifols.

The resulting suspension was centrifuged at 140 g for 2 minutes. Excess hydrating solution was removed using a micropipette. The hydrated biocompatible inorganic materials were combined with the hydrogels outlined in Table 2 with manual mixing. The resulting compositions are outline in Table 3. The physical characteristics of the biocompatible inorganic materials are listed below for reference:

| | |
|---|---|
| β-TCP (Tricalcium | <150 μm; helium density 3.016 g/cm³; |
| phosphate) | Calcium:Phosphate (1.4-1.5) |
| β-TCP/Hydroxyapatite | 25% β-TCP, 75% Hydroxyapatite; <150 μm; |
| | Calcium:Phosphate (1.4-1.5) |
| Bioglass | −200 μm; 90% <100 μm |

The #1-9 numbering utilised in Table 3 directly corresponds to the numbering of the bone constructs illustrated in FIG. 1.

TABLE 3

| | #1 | #2 | #3 | #4 | #5 | #6 | #7 | | #8 | | #9 | |
|---|---|---|---|---|---|---|---|---|---|---|---|---|
| Alginate | — | — | — | — | — | — | 1 | mL | 1 | mL | 1 | mL |

| Hydrogel | | | | | | | | | | | | |
|---|---|---|---|---|---|---|---|---|---|---|---|---|
| Gelatin | — | — | — | 1 | mL | 1 | mL | 1 | mL | — | — | — |

| Hydrogel | | | | | | | | | | | | |
|---|---|---|---|---|---|---|---|---|---|---|---|---|
| Hyaluronic | 1 | mL | 1 | mL | 1 | mL | — | — | — | — | — | — |

| acid | | | | | | | | | | | | |
|---|---|---|---|---|---|---|---|---|---|---|---|---|
| Hydrogel | | | | | | | | | | | | |
| Bioglass | — | — | 3.143 | g | — | — | 3.143 | g | — | — | 3.143 | g |

| | | | | | | | | | | | | |
|---|---|---|---|---|---|---|---|---|---|---|---|---|
| β-TCP | 5.250 | g | — | — | 5.250 | g | — | — | 5.250 | g | — | — |

| | | | | | | | | | | | | |
|---|---|---|---|---|---|---|---|---|---|---|---|---|
| HA* + | — | 4.703 | g | — | — | 4.703 | g | — | — | 4.703 | g | — |

β-TCP

* = 75% Hydroxyapatite (HA): 25% β-TCP

3D Printing processes for printing the compositions outlined in Tables 1 and 3 all follow a generalised methodology. The fibrinogen and thrombin parts are printed as alternating layers. The number of layers printed is at the operators discretion, but typically ranges between 2-20. Once the desired number of fibrinogen and thrombin layers are printed, the paste composition is printed on top of the layered fibrinogen/thrombin structure.

Taking third part composition #7 (Alginate & β-TCP) as an example, utilising a 3D printer, the fibrinogen component and thrombin component (Table 1) were printed alternately from separate containers as follows. A first layer of the fibrinogen component with a volume of 0.0177 cm³ (17.7 μL) was printed onto the printing surface area. A second layer of thrombin with a volume of 0.0177 cm³ (17.7 μL) is printed on top of the previous layer of fibrinogen. A total of 8 layers of fibrinogen interleaved with 8 layers of thrombin are printed to generate an alternating layered structure.

Subsequently, 0.149 cm³ (149 μL) of third part composition #7 outlined in Table 3 was printed on top of the alternating layered structure of fibrinogen and thrombin, again this material was printed from a separate container. The resulting structure comprising the three compositions printed onto one another is termed the first macrolayer. In this macrolayer, the overall ratio of the fibrinogen composition to thrombin composition to composition #7 is approximately 1:1:1. Within the macrolayer, the fibrinogen and thrombin layers react to form fibrin and strengthen the structure.

Further macrolayers are printed adjacent to, and on top of the first macrolayer in a sequential order according to a design file to build-up the bone constructs outlined in FIG. 1.

During the printing process, the shear rate in the syringe tip acting on the compositions was determined to be 15 and 40 s⁻¹.

Prior to printing an upper macrolayer directly on-top of a lower macrolayer the operator may perform the additional step of printing a base layer on top of the lower macrolayer. The base layer typically consists of a single layer of fibrinogen and a single layer of thrombin. The perimeter of the base layer projects upward to define an open space bounded by an upwardly turned perimeter. The base layer in effect functions and appears like a nest. The upper macrolayer is printed (as discussed supra) into the open space defined in the base layer, and the upwardly turned perimeter provides a nesting function that adds structural support to the upper macrolayer once it is printed.

For the avoidance of any doubt, the concept of printing a base layer that functions like a nest so as to provide structure and support to further layers printed therein, is a general concept applicable to and combinable with all method steps of the present invention. The concept should be not treated as being isolated to the particular example discussed in the preceding paragraphs. The skilled person should appreciate the general applicability of the base or nest layer to the methods of the invention.

### EXAMPLE 2

### Compatibility of the Compositions of the Invention With Human Mesenchymal Stem Cells

The ability of the compositions of the present invention to provide a non-toxic environment to cells, proteins and other bioactive materials is highly advantageous. 3D Printed bone constructs that facilitate cell survival and cell differentiation are highly desirable from a therapeutic perspective.

Human mesenchymal stem cells (hMSC) were obtained from the bone marrow of human donors. The hMSC were extracted in accordance with GMP practices, and subsequently expanded ex vivo in Dulbecco's Modified Eagle Medium (DMEM) and 10% Human Serum B (hSERB). The cell culture medium was centrifuged, and any excess liquid was removed. The resulting cells formed sediment at the bottom of the centrifuge tube.

The sediment of hMSC was mixed with compositions #1, #2, #7, and #8 shown in Table 3. The hMSC sediment was mixed with the relevant hydrogel and the resultant mixture was added to the hydrated biocompatible inorganic material. The resultant paste was manually mixed (gently) to avoid any cell damage. The resultant compositions are outlined in Table 4 with the numbering #1′, #2′, #7′ and #8′ for ease of reference.

TABLE 4

| | #1′ | | #2′ | | #7′ | | #8′ | |
|---|---|---|---|---|---|---|---|---|
| Alginate | — | | — | | 1 | mL | 1 | mL |
| Gelatin | — | | — | | — | | — | |
| Hyaluronic | 1 | mL | 1 | mL | — | | — | |
| acid | | | | | | | | |
| Bioglass | — | | — | | — | | — | |
| β-TCP | 5.250 | g | — | | 5.250 | g | — | |
| HA* + β- | — | | 4.703 | g | | | 4.703 | g |
| TCP | | | | | | | | |
| hMSC | 4 × 10⁶ | cells | 4 × 10⁶ | cells | 4 × 10⁶ | cells | 4 × 10⁶ | cells |

*= 75% Hydroxyapatite (HA):25% β-TCP

Compositions #1′, #2′, #7′ and #8′ including the hMSC were mixed with the fibrinogen and thrombin components of Table 1 in-vitro (without 3D printing) in an approximate ratio of 1:1:1 by volume. The resultant mixture was cultured in the standard conditions discussed supra (DMEM+10% hSERB) for 7 days.

CELLTITER-GLO 3D Cell Viability Assay (Promega) was used to determine the number of viable cells in 3D cell culture based on quantification of ATP, a marker for the presence of metabolically active cells. After cell lysis the luminescent signal obtained was proportional to the concentration of ATP present in the sample and therefore to the number of viable cells present in culture.

Briefly, an equivalent volume of CELLTITER-GLO 3D Reagent (Promega) was added to the cell culture volume and the contents were mixed vigorously for 5 minutes to induce cell lysis. The plate was incubated at room temperature for an additional 25 minutes to stabilize the luminescent signal to be recorded.

FIG. 2 illustrates the results of the cell viability assay. From FIG. 2 it is evident that each of compositions #1′, #2′, #7′ and #8′ showed an increase in the ATP concentration at days 3 and 7. Composition #7′ depicted particularly promising results, although all the compositions tested gave positive outcomes. Thus, it can be concluded that the compositions are non-toxic to the hMSC.

The compositions outlined in Table 4 were also subjected to a LIVE/DEAD Cell Viability Assay (Life Technologies) based on the simultaneous detection of live and dead cells by employing two probes that respectively measure intracellular esterase activity (by calcein AM probe) and plasma membrane integrity (by ethidium homodimer (EthD-1) probe). The nuclear content was stained with Hoechst 33342 fluorescent dye. A fluorescence microscope was used to visualize cell characteristics.

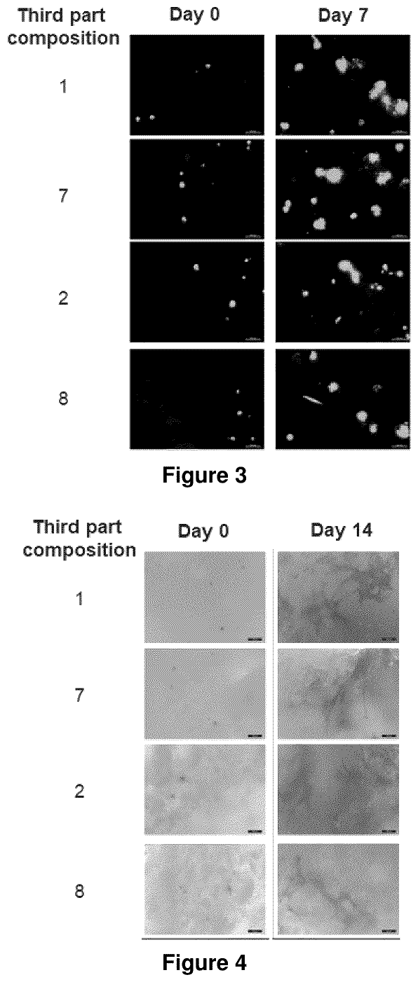

In FIG. 3, we see images from the cell viability assay captured with a 10× objective at days 0 and 7. The figure shows viable hMSC, dead hMSC, and stained nuclei. At day 0 there is very little to note in relation to any of the compositions. However, at day 7 the large shaded, blotched areas show that a high concentration of cells are viable when mixed with the compositions of the present invention. Thus, from FIG. 3 it is clear that the compositions of the invention are suitable to permit cell adhesion and spatial cell distribution throughout a bone scaffold as well as to promote cell growth.

### EXAMPLE 3

### Determining the Osteogenic Potential of the Compositions of the Invention

The osteogenic potency of the compositions outlined in Table 4 was determined by a cell differentiation analysis protocol.

Compositions #1′, #2′, #7′ and #8′ of Table 4 including the hMSC were mixed with the fibrinogen and thrombin components of Table 1 in-vitro (without 3D printing) in an approximate ratio of 1:1:1 by volume. The volume of the resultant mixture was supplemented with an equal volume of osteogenic differentiation culture media (STEMPRO Basal Media (90%)+STEMPRO Osteo Supplement 10% from GIBCO). The osteogenic differentiation culture media was replaced every 3 days.

At day 14 the compositions/hMSC were were tested for alkaline phosphatase (ALP) activity using SIGMAFAST BCIP/NBT solution. The compositions before differentiation culture showed no ALP staining (see Day 0, FIG. 4). However, at Day 14 a large number of cells stained positive for ALP activity, as illustrated by the filament type structures stained under Day 14 in FIG. 4. FIG. 4 illustrates a high number of ALP positive cells, for each of compositions #1′, #2′, #7′ and #8′, after 14 days of osteogenic culturing. These findings positively reinforce that the compositions of the present invention support successful differentiation/osteoinduction of hMSC into osteoblasts. Moreover, the cells displayed osteocyte-like morphology upon visual inspection at day 14.

All of the compositions assayed facilitated the differentiation of the hMSC into osteocytes.

### EXAMPLE 4

### Printing Objects of a Defined Geometry Using a Two-Part Composition of the Present Invention

Two part compositions within the scope of the present invention were prepared according to Table 5. Depending upon whether the second composition contained fibrinogen or thrombin, the first composition consisted of the other of fibrinogen or thrombin as outlined in Table 1 supra. The second compositions contained the components outlined in Table 5.

TABLE 5

| | #A | #B | #C | #D | |
|---|---|---|---|---|---|
| Alginate | — | — | — | 1 | mL |

| Hydrogel | | | | | | | |
|---|---|---|---|---|---|---|---|
| Hyaluronic | 1 | mL | 1 | mL | 1 | mL | — |

| Acid | | | | | | | |
|---|---|---|---|---|---|---|---|
| Hydrogel | | | | | | | |
| Bioglass | — | 3.143 | g | 3.143 | g | 3.143 | g |

| | | | | | |
|---|---|---|---|---|---|
| β-TCP | 5.250 | g | — | — | — |

| | | | | | | |
|---|---|---|---|---|---|---|
| Fibrinogen | — | 5 | mL | — | 5 | mL |

| Composition | | | | | | |
|---|---|---|---|---|---|---|
| Table 1 | | | | | | |
| Thrombin | 5 | mL | — | 5 | mL | — |

Composition
Table 1

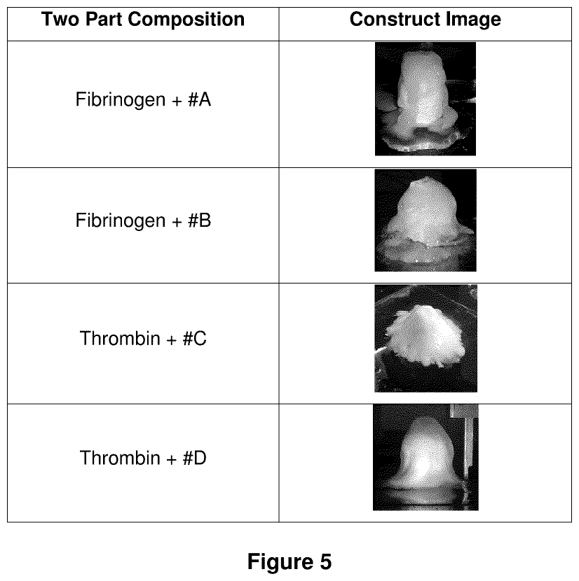

As illustrated in FIG. 5, the two part compositions of the present invention do not 3D print with the level of control or accuracy of the three part compositions of the present invention. However, promising results were obtained for compositions #A, #B, and #D, and Example 4 serves as a proof of concept study that suitable bone constructs can be 3D printed with a two part composition within the scope of the present invention and that the accuracy can be finessed with further diligence and time.

1. A multi-part composition for 3-dimensional printing of a bone composite, the multi-part composition comprising:
i) a first part comprising fibrinogen in a pharmaceutically acceptable carrier;
ii) a second part comprising thrombin in a pharmaceutically acceptable carrier; and
iii) a third part comprising a pharmaceutically acceptable hydrogel mixed with at least one biocompatible inorganic material, the at least one biocompatible inorganic material providing a source of at least one of calcium and phosphorous atoms, wherein the third part has an apparent viscosity selected from the group consisting of:
from about 1 to about 300 Pa s at a shear rate of 0.1 s⁻¹ as measured by a rotational viscometer at 25° C. and 1 atm of pressure, and
from about 1 to about 100 Pa s at a shear rate of 1 s⁻¹ as measured by a rotational viscometer at 25° C. and 1 atm of pressure,
and further wherein the third part is a standalone composition, or it is mixed with either the first part or the second part of the multipart composition, such that at least the first and second parts of the multi-part composition do not mix prior to printing by a 3-dimensional printing device.

2. The composition according to claim 1, wherein in said first part the fibrinogen is at a concentration between about 5 to about 200 mg/mL.

3. The composition according to claim 1, wherein in said second part the thrombin is at a concentration about 25 and about 1500 IU/mL.

4. The composition according to claim 1, wherein the third part of the multipart composition has an apparent viscosity selected from the group consisting of:
from about 50 to about 250 Pa·s at a shear rate of 0.1 s⁻¹ as measured by rotational viscometer at room temperature and pressure, and
from about 2 to about 50 Pa·s at a shear rate of 1 s⁻¹ as measured by rotational viscometer at room temperature and pressure.

5. The composition according to claim 1, wherein said third part is a standalone composition such that the three parts of the multipart composition do not mix prior to printing by a 3-dimensional printing device.

6. The composition according to claim 1, wherein said biocompatible inorganic material is selected from the group consisting of bioglass, tricalcium phosphate, single-phase hydroxyapatite, biphasic hydroxyapatite-tricalcium phosphate, natural bone powder, and combinations thereof.

7. The composition according to claim 1, wherein said biocompatible inorganic material is selected from the group consisting of tricalcium phosphate, single-phase hydroxyapatite, biphasic hydroxyapatite-tricalcium phosphate, and combinations thereof.

8. The composition according to claim 1, wherein the biocompatible inorganic material is beta-tricalcium phosphate.

9. The composition according to claim 8, wherein the beta-tricalcium phosphate has a density of between about 2.95 g/cm³ and about 3.15 g/cm³ as determined by helium pycnometry.

10. The composition according to claim 8, wherein the beta-tricalcium phosphate has a density of between about 2.95 g/cm³ and about 3.10 g/cm³ as determined by helium pycnometry.

11. The composition according to claim 8, wherein the beta-tricalcium phosphate has a d90 particle size distribution of not more than about 180 μm.

12. The composition according to claim 8, wherein the beta-tricalcium phosphate has a d90 particle size distribution of not more than about 160 μm.

13. The composition according to claim 8, wherein the beta-tricalcium phosphate is characterized by an X-ray powder diffraction pattern comprising unique peaks at °2θ (d value Å); angles of 17.0 (5.2), 21.9 (4.1), 25.8 (3.45), 27.8 (3.2), 29.65 (3.0), 31.0 (2.9), 32.45 (2.75), 34.4 (2.6), 46.9 (1.9), 48.0 (1.9), 48.4 (1.9), and 53.0 (1.7) when obtained with a Cu tube anode with K-alpha radiation.

14. The composition according to claim 1, wherein said biocompatible inorganic material has an average particle size of less than about 150 μm.

15. The composition according to claim 1, wherein said biocompatible inorganic material has an average particle size of less than about 100 μm.

16. (canceled)

17. (canceled)

18. (canceled)

19. (canceled)

20. (canceled)

21. (canceled)

22. (canceled)

23. (canceled)

24. (canceled)

25. A bone composite obtainable from the multi-part composition according to claim 1.

26. A bone composite according to claim 25, wherein said biocompatible inorganic material is at a concentration of between about 5% w/w to about 60% w/w of the bone composite.

27. A method for preparing a bone composite, the method comprising:
i) providing a 3-dimensional printing device with the multi-part composition of claim 1;
ii) printing the bone composite according to a determined design; and
iii) optionally incubating the bone composite.

28. The method of claim 27, wherein the 3-dimensional printing device prints,
i) a first layer comprising either fibrinogen or thrombin;
ii) a second layer comprising either fibrinogen or thrombin;
wherein if the first layer comprises fibrinogen the second layer comprises thrombin and vice versa,
iii) sequentially repeating said i) and ii) n times, wherein n≥1, to generate an alternating layered structure;
iv) printing the third part comprising the hydrogel and biocompatible inorganic material on top of the alternating layered structure of said iii); and
v) optionally repeating said i)-iv) as necessary to provide the bone composite.

29. The method of claim 28, wherein the fibrinogen is printed first, and the thrombin is printed second as a layer on top of the fibrinogen.

30-43. (canceled)
